(19) United States
(12) Patent Application Publication
(10) Pub. No.: US 2020/0188684 A1
(43) Pub. Date: Jun. 18, 2020

(54) Polymer Introducer for Use with an RF Ablation Probe and Associated RF Ablation Probe Assembly

(71) Applicant: Avent, Inc., Alpharetta, GA (US)

(72) Inventors: Ruoya Wang, Decatur, GA (US); Ryan Smith, Atlanta, GA (US); Ken Driver, Brookhaven, GA (US); Samir Merchant, Cumming, GA (US)

(21) Appl. No.: 16/220,169

(22) Filed: Dec. 14, 2018

(51) Int. Cl. A61N 1/40; A61M 25/06; A61B 18/14; A61B 17/34

(52) U.S. Cl. CPC A61N 1/403; A61M 25/0662; A61F 2007/126; A61B 17/3421; A61B 18/1477

(57) ABSTRACT

A medical introducer is provided for use in locating an energy delivery probe, such as an RF ablation probe, at a target location in tissue. The introducer includes a proximal hub and an elongate cannula having a distal end and a proximal end, the proximal end connected to the hub. The cannula is formed entirely of an electrically insulating polymer material and includes a tapered distal tip. An RF ablation probe system is also provided that includes an RF ablation probe assembly, a stylet, and the introducer.

### FIELD OF THE INVENTION

The present invention relates generally to a medical cannula or introducer, and more particularly to an introducer used to guide a probe for applying energy for the treatment of tissue, for example in an RF ablation procedure.

### BACKGROUND

Lower back injuries and chronic joint pain are major health problems resulting not only in debilitating conditions for the patient, but also in the consumption of a large proportion of funds allocated for health care, social assistance and disability programs. In the lower back, disc abnormalities and pain may result from trauma, repetitive use in the workplace, metabolic disorders, inherited proclivity, and/or aging. The existence of adjacent nerve structures and innervation of the disc are very important issues in respect to patient treatment for back pain. In joints, osteoarthritis is the most common form of arthritis pain and occurs when the protective cartilage on the ends of bones wears down over time.

A minimally invasive technique of delivering high-frequency electrical current has been shown to relieve localized pain in many patients. Generally, the high-frequency current used for such procedures is in the radiofrequency (RF) range, i.e. between 100 kHz and 1 GHz and more specifically between 300-600 kHz. The treatment of pain using high-frequency electrical current has been applied successfully to various regions of patients' bodies suspected of contributing to chronic pain sensations. In addition to creating lesions in neural structures, application of radiofrequency energy has also been used to treat tumors throughout the body.

The RF electrical current is typically delivered from a generator via connected electrodes that are placed in a patient's body, in a region of tissue that contains a neural structure suspected of transmitting pain signals to the brain. The electrodes generally include an insulated shaft with an exposed conductive tip to deliver the radiofrequency electrical current. Tissue resistance to the current causes heating of tissue adjacent resulting in the coagulation of cells (at a temperature of approximately 45° C. for small unmyelinated nerve structures) and the formation of a lesion that effectively denervates the neural structure in question. Denervation refers to affecting a neural structure's ability to transmit signals and usually results in the complete inability of a neural structure to transmit signals, thus removing the pain sensations.

To extend the size of a lesion, radiofrequency treatment may be applied in conjunction with a cooling mechanism, whereby a cooling means is used to reduce the temperature of the tissue near an energy delivery device, allowing a higher voltage to be applied without causing an unwanted increase in local tissue temperature. The application of a higher voltage allows regions of tissue further away from the energy delivery device to reach a temperature at which a lesion can form, thus increasing the size/volume of the lesion. In addition, radiofrequency ablation relies on the application of electrical energy to create heat tissue based on a closed-loop temperature feedback routine. More specifically, the ablation routine applies radiofrequency energy to reach and maintain preset temperature profiles. The temperature is typically measured through a thermocouple located at the distal tip of the active electrode. The output from the thermocouple must be filtered to reject the radiofrequency frequency prior to amplification.

Various procedures using RF probes for pain management or treatment use a cannula or “introducer” with a stylet to puncture the patient's skin and create a pathway to the target nerve location. Once the introducer is placed, the stylet is withdrawn from the introducer and the RF probe is inserted through the lumen in the introducer and secured to a proximal hub on the introducer, for example using a luer-lock fitting so that the active distal end of the probe extends beyond the distal end of the introducer. These introducers may also include a radiopaque band, such as a platinum band, at the distal end thereof and/or radiopaque markings along the length of the introducer visual lesion location under fluoroscopy.

U.S. Pat. No. 8,821,406 describes a polyethylene introducer for a vascular temperature probe assembly wherein the introducer hub includes a side port for infusion of fluids into the hub and through the lumen in the introducer. However, with this device, the probe is snug within the introducer and there is little room for infusion of liquids through the introducer with the probe in place. The '406 patent describes to remove the probe from the introducer to facilitate the injection of fluids through the side port in the introducer hub.

Conventional introducers for use in ablation procedures are typically made of a high strength metal, such as 304 grade stainless steel, which is expensive and adds to the overall cost of the probe kits and procedures. It has been the general belief that the use of such metals is necessary for their resistance to bending forces to prevent kinking of the introducer during the skin puncture and tissue penetration process while maintaining a thin-wall structure to minimize trauma to the tissue.

Also, the manufacturing process for the conventional introducers is relatively complex and labor intensive, which also adds to the overall costs of the introducer and treatment procedures. The introducer is used to establish the active tip length of the probe by creating an electrical barrier along the length of the probe shaft, wherein only the portion of the probe not covered by the introducer is electrically exposed and delivers RF energy into the tissue. For this reason, the metal introducer must be covered with an electrically insulating material, which is typically done by bonding a polymer polyamide material to the introducer blank. The polyamide creates a strong electrical barrier, but must be subsequently trimmed and bonded to the metal shaft with a relatively long cure time adhesive.

Thus, a new and improved introducer that addresses the issues noted above would be welcomed in the art.

### SUMMARY OF THE INVENTION

Objects and advantages of the invention will be set forth in part in the following description, or may be obvious from the description, or may be learned through practice of the invention.

In one aspect, the present invention is directed to a medical introducer for use in locating an energy delivery probe at a target location in tissue. For example, the introducer may be used with an RF ablation probe in performance of an RF ablation procedure to manage or treat pain. The introducer includes a proximal hub, and an elongate cannula having a distal end and a proximal end, the proximal end connected to the hub. The cannula is formed entirely of an electrically insulating polymer material. The cannula does not have a metal core or sleeve surrounded by a polymer material, but is formed homogeneously of the polymer material throughout. The cannula further includes a tapered distal tip.

In certain embodiments, the polymer material is selected to have a flexibility that allows the cannula to bend without kinking during insertion of the introducer into the tissue. The polymer material may be one of nylon, fluorinated ethylene propylene (FEP), polytetrafluoroethylene (PTFE), high-density polyethylene (HDPE), polyamide, or polyether ether ketone (PEEK).

The hub may be overmolded onto the proximal end of the polymer cannula or, in an alternate embodiment, the hub may be bonded onto the proximal end of the cannula with a compatible adhesive.

For cooled ablation procedures, the introducer may include a fluid introduction port in fluid communication with the proximal end of the cannula, wherein fluids such as saline or a local anesthesia can be injected into the target tissue via the port and the cannula without removing the probe from the cannula.

In other embodiments, a radiopaque element is fixed to the distal end of the cannula and defines the tapered distal tip. The radiopaque element may be co-extruded with the cannula, coated onto the cannula, or attached with an adhesive or other suitable fixation means. In addition a plurality of radiopaque bands may applied over the polymer material and spaced apart along the elongate cannula.

In other aspects, the invention encompasses an RF ablation probe system for use in locating a radio frequency (RF) probe assembly at a target location in tissue to treat or manage pain in a patient. The system includes the introducer discussed above, as well as a stylet that is insertable through the hub and into the cannula. The stylet includes a tissue-piercing distal end that extends from the distal end of the cannula when the stylet is inserted into the introducer. The stylet includes an outside diameter such that a concentric clearance between the stylet and the inside diameter of the cannula is less than 0.0010 inches. The system also includes an RF probe assembly with an elongate shaft that insertable through the hub (with the stylet removed from the introducer) and into the cannula, the elongate shaft has an active distal tip that extends from the distal end of the cannula when the elongate shaft is inserted into the introducer. The elongate shaft has an outside diameter such that a concentric clearance between the RF probe shaft and the inside diameter of the cannula is greater than 0.0010 inches.

In a particular embodiment, the concentric clearance between the stylet and the inside diameter of the cannula is less than 0.0007+/−0.0002 inches, and may be for example 0.0005 inches. The concentric clearance between the RF probe shaft and the inside diameter of the cannula may be 0.0020+/−0.0010 inches, such as 0.0020 inches.

The other characteristics of the introducer discussed above are applicable to the introducer included with the RF ablation probe system.

The present invention also encompasses various method embodiments for use of the introducer and RF ablation system as described and enabled above.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a diagram of a system for applying radiofrequency (RF) electrical energy to target tissue in a patient's body;

FIG. 2 is a perspective view of one embodiment of an introducer and stylet in accordance with the present disclosure;

FIG. 3 is a longitudinal cross-section of the combined stylet and introducer of FIG. 2;

FIGS. 4a and 4b are enlarged cross-sectional views of the distal end of the stylet and the RF probe shaft within the introducer cannula, particularly illustrating dimensional characteristics according to the present disclosure; and

FIG. 5 is an enlarged view of the distal end of the introducer, particularly illustrating the tapered distal tip of the cannula.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

Before explaining various embodiments of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of this invention, a lesion refers to any effect achieved through the application of energy to a tissue in a patient's body, and the invention is not intended to be limited in this regard. Furthermore, for the purposes of this description, proximal generally indicates that portion of a device or system next to or nearer to a user (when the device is in use), while the term distal generally indicates a portion further away from the user (when the device is in use).

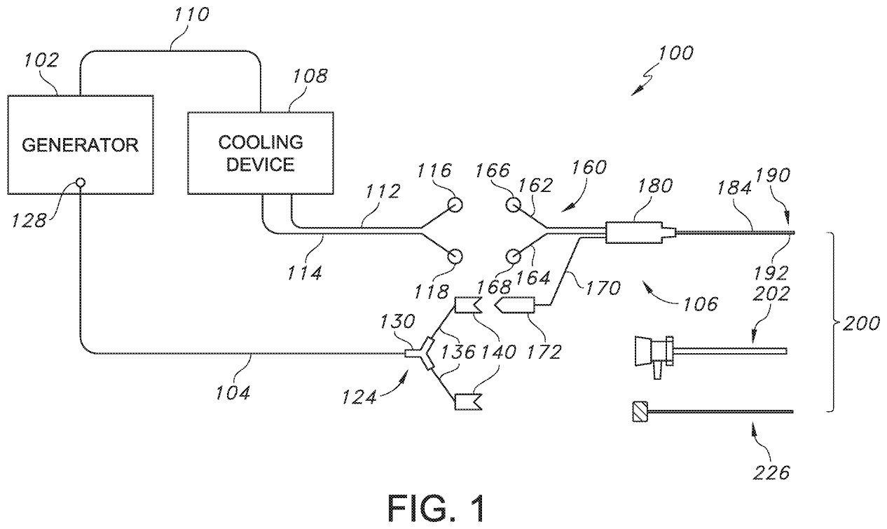

Referring now to the drawings, FIG. 1 illustrates a schematic diagram of a system 100 for application of energy, such as RF energy, to a target location within tissue of a patient, and is presented herein for purposes of describing an exemplary operating environment in which the present introducer and assembly may be used. The system 100 includes a generator 102, a cable 104, first and second probe assemblies 106 (only one probe assembly is shown), one or more cooling devices 108, a pump cable 110, one or more proximal cooling supply tubes 112, and one or more proximal cooling return tubes 114. The generator 102 may be a radiofrequency (RF) generator, or any other energy source, such as microwave energy, thermal energy, ultrasound, or optical energy. The generator 102 may include a display that displays various aspects of a treatment procedure, such as any parameters that are relevant to a treatment procedure, for example temperature, impedance, etc. and errors or warnings related to a treatment procedure. Alternatively, the generator 102 may include means of transmitting a signal to an external display. The generator 102 is operable to communicate with the first and second probe assemblies 106 and the one or more cooling devices 108. Such communication may be unidirectional or bidirectional depending on the devices used and the procedure performed.

In addition, as shown, a distal region 124 of the cable 104 may include a splitter 130 that divides the cable 104 into two distal ends 136 such that the probe assemblies 106 can be connected thereto. A proximal end 128 of the cable 104 is connected to the generator 102. This connection can be permanent, whereby, for example, the proximal end 128 of the cable 104 is embedded within the generator 102, or temporary, whereby, for example, the proximal end 128 of cable 104 is connected to generator 102 via an electrical connector. The two distal ends 136 of the cable 104 terminate in connectors 140 operable to couple to the probe assemblies 106 and establish an electrical connection between the probe assemblies 106 and the generator 102. In alternate embodiments, the system 100 may include a separate cable for each probe assembly 106 being used to couple the probe assemblies 106 to the generator 102.

The cooling device(s) 108 may include any means of reducing a temperature of material located at and proximate to one or more of the probe assemblies 106. For example, the cooling devices 108 may include a pump assembly operable to circulate a fluid from the cooling devices 108 through one or more proximal cooling supply tubes 112, the probe assemblies 106, one or more proximal cooling return tubes 114, and back to the one or more cooling devices 108.

The system 100 may include a controller for facilitating communication between the cooling devices 108 and the generator 102 via a feedback control loop. The feedback control may be implemented, for example, in a control module which may be a component of the generator 102. In such embodiments, the generator 102 is operable to communicate bi-directionally with the probe assemblies 106 as well as with the cooling devices 108, wherein bi-directional communication refers to the capability of a device to both receive a signal from and send a signal to another device.

As an example, the generator 102 may receive temperature measurements from one or both of the first and second probe assemblies 106. Based on the temperature measurements, the generator 102 may perform some action, such as modulating the power that is sent to the probe assemblies 106. Thus, both probe assemblies 106 may be individually controlled based on their respective temperature measurements.

The pumps associated with the cooling devices 108 may communicate a fluid flow rate to the generator 102 and may receive communications from the generator 102 instructing the pumps to modulate this flow rate. With the cooling devices 108 turned off, any temperature sensing elements associated with the probe assemblies 106 would not be affected by the cooling fluid allowing a more precise determination of the surrounding tissue temperature to be made. In addition, when using more than one probe assembly 106, the average temperature or a maximum temperature in the temperature sensing elements associated with probe assemblies 106 may be used to modulate cooling.

The cooling devices 108 may reduce the rate of cooling or disengage depending on the distance between the probe assemblies 106. For example, when the distance is small enough such that a sufficient current density exists in the region to achieve a desired temperature, little or no cooling may be required. In such an embodiment, energy is preferentially concentrated between first and second energy delivery devices 192 through a region of tissue to be treated, thereby creating a strip lesion characterized by an oblong volume of heated tissue that is formed when an active electrode is in close proximity to a return electrode of similar dimensions.

The cooling devices 108 may also communicate with the generator 102 to alert the generator 102 to one or more possible errors and/or anomalies associated with the cooling devices 108. For example, if cooling flow is impeded or if a lid of one or more of the cooling devices 108 is opened. The generator 102 may then act on the error signal by at least one of alerting a user, aborting the procedure, and modifying an action.

Still referring to FIG. 1, the proximal cooling supply tubes 112 may include proximal supply tube connectors 116 at the distal ends of the one or more proximal cooling supply tubes 112. Additionally, the proximal cooling return tubes 114 may include proximal return tube connectors 118 at the distal ends of the one or more proximal cooling return tubes 114. In one embodiment, the proximal supply tube connectors 116 are female luer-lock type connectors and the proximal return tube connectors 118 are male luer-lock type connectors although other connector types are intended to be within the scope of the present invention.

In addition, as shown in FIG. 1, the probe assembly 106 may include a proximal region 160, a handle 180, a hollow elongate shaft 184, and a distal tip region 190 that includes the one or more energy delivery devices 192. The elongate shaft 184 may be manufactured out of polyimide, which provides exceptional electrical insulation while maintaining sufficient flexibility and compactness. In alternate embodiments, the elongate shaft 184 may be any other material imparting similar qualities. In still other embodiments, the elongate shaft 184 may be manufactured from an electrically conductive material and may be covered by an insulating material so that delivered energy remains concentrated at the energy delivery device 192 of the distal tip region 190. The proximal region 160 includes a distal cooling supply tube 162, a distal supply tube connector 166, a distal cooling return tube 164, a distal return tube connector 168, a probe assembly cable 170, and a probe cable connector 172. In such embodiments, the distal cooling supply tube 162 and distal cooling return tube 164 are flexible to allow for greater maneuverability of the probe assemblies 106, but alternate embodiments with rigid tubes are possible.

The distal supply tube connector 166 may be a male luer-lock type connector and the distal return tube connector 168 may be a female luer-lock type connector. Thus, the proximal supply tube connector 116 may be operable to interlock with the distal supply tube connector 166 and the proximal return tube connector 118 may be operable to interlock with the distal return tube connector 168.

The probe cable connector 172 may be located at a proximal end of the probe assembly cable 170 and may be operable to reversibly couple to one of the connectors 140, thus establishing an electrical connection between the generator 102 and the probe assembly 106. The probe assembly cable 170 includes one or more conductors to transmit RF current from the generator 102 to the one or more energy delivery devices 192, as well as to connect multiple temperature sensing devices to the generator 102 as discussed below.

The energy delivery devices 192 may include any means of delivering energy to a region of tissue adjacent to the distal tip region 190. For example, the energy delivery devices 192 may include an ultrasonic device, an electrode or any other energy delivery means and the invention is not limited in this regard. Similarly, energy delivered via the energy delivery devices 192 may take several forms including but not limited to thermal energy, ultrasonic energy, radiofrequency energy, microwave energy or any other form of energy. For example, in one embodiment, the energy delivery devices 192 may include an electrode. The active region of the electrode may be 2 to 20 millimeters (mm) in length and energy delivered by the electrode is electrical energy in the form of current in the RF range. The size of the active region of the electrode can be optimized for placement within an intervertebral disc, however, different sizes of active regions, all of which are within the scope of the present invention, may be used depending on the specific procedure being performed. In some embodiments, feedback from the generator 102 may automatically adjust the exposed area of the energy delivery device 192 in response to a given measurement such as impedance or temperature. For example, in one embodiment, the energy delivery devices 192 may maximize energy delivered to the tissue by implementing at least one additional feedback control, such as a rising impedance value.

FIG. 1 also depicts an introducer 202 and a stylet 226, wherein the combination of the RF probe assembly 106, the introducer 202, and the stylet 226 define an RF ablation probe system 200 in accordance with aspects of the present invention.

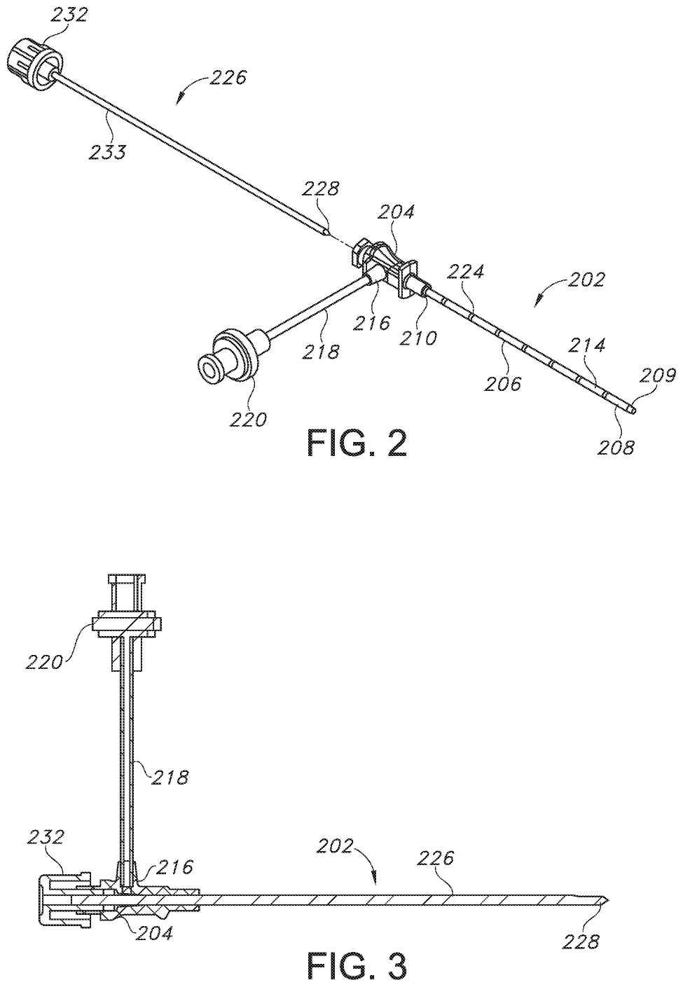

Referring to FIGS. 2 and 3, generally, the introducer 202 has a proximal end 210 configured with a hub 204 and a cannula 206 (defining an internal lumen) having a distal end 208. As understood in the art, the introducer 202 is operable to easily and securely couple with the RF probe assembly 106. For example, the proximal hub 204 is configured with a connector, such as a luer-lock connector, able to mate with the handle 180 of the RF probe assembly 106. The introducer cannula 206 is used to gain access to a tissue treatment site within a patient's body, wherein the elongate shaft 184 of the RF probe assembly 106 may be introduced to the treatment site through the longitudinal lumen of the introducer cannula 206.

Function of the stylet 226 is understood in the art. Generally, the stylet 226 includes a proximal hub 232 fixed to an elongate needle 233 having a beveled tip at the distal end 228 thereof. The elongate needle slides through the introducer 202 such that the stylet hub 232 connects to the introducer hub 204, for example via a luer-lock connection between the hubs 232 and 204, as depicted in FIG. 3. The distal end 228 of the stylet needle 233 extends past the distal end 208 of the introducer cannula 206 to facilitate insertion of the introducer cannula 206 into the patient's body at the treatment target site. Various forms of stylets 226 are well known in the art and the present invention is not limited to include only one specific form. Further, the stylet 226 may be operable to connect to a power source and may therefore form part of an electrical current impedance monitor.

Referring to FIGS. 2 through 5, the cannula 206 component of the introducer 202 is formed entirely of an electrically insulating polymer material 214. The cannula does not have a metal core or metal sleeve that is surrounded by a polymer material, but is formed homogeneously of the polymer material 214 throughout. The polymer material 214 is selected to have a flexibility that allows the cannula 206 to bend without kinking during insertion of the introducer 202 into the tissue. In certain embodiments, the polymer material may be one of nylon, fluorinated ethylene propylene (FEP), polytetrafluoroethylene (PTFE), high-density polyethylene (HDPE), polyamide, or polyether ether ketone (PEEK). The polymer material has a sufficiently high dielectric strength to act as an insulator.

Use of the polymer material 214 allows for the plastic proximal hub 204 to be overmolded directly onto the proximal end 210 of the polymer cannula 206. In other embodiments, the hub 204 may be bonded onto the proximal end 210 of the cannula 206 with a compatible adhesive.

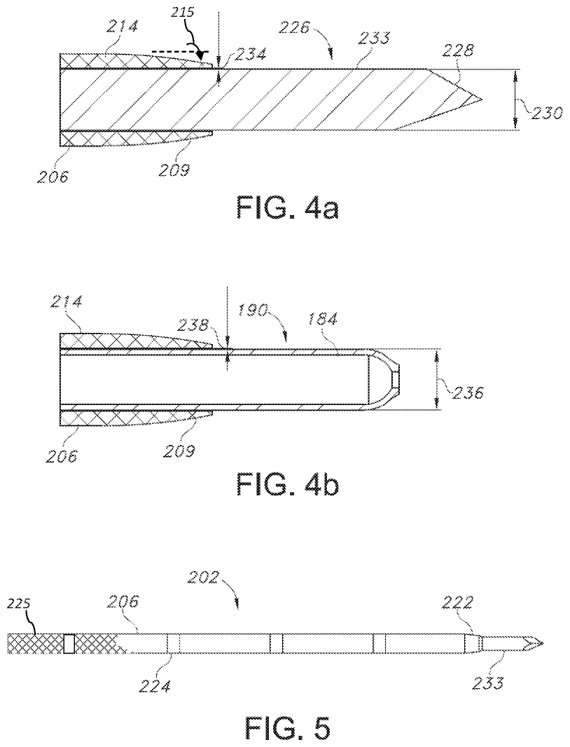

Use of the polymer material 214 to form the cannula 206 eliminates the prior practice of coating a metal cannula with polyamide to render the cannula non-conductive followed by the tedious need to trim the polyamide. Contrary to conventional thought, it has been found that reliance of an expensive metal cannula is not necessary, and that the stylet 226 can provide sufficient strength and rigidity to the combination of elements during the tissue piercing and insertion procedure, particularly if a relatively tight fit is maintained between the stylet needle 233 and the inner diameter of the cannula 206. For example, referring to FIG. 4a, in one embodiment the needle component 233 of the stylet 226 includes an outside diameter 230 such that a concentric clearance 234 between the needle 233 and the inside diameter of the cannula 206 is less than 0.0010 inches. This concentric clearance 234 may be, for example, 0.0007+/−0.0002 inches. In a particular embodiment, the concentric clearance is 0.0005 inches. It should be appreciated that, with this relatively small degree of concentric clearance 234, the possibility of peel-back, kinking, or damage of the distal end 208 of the polymer cannula 226 is minimized during the tissue insertion procedure. However, fluid delivery through the fluid delivery port 216 (discussed in greater detail below) will be subjected to high back pressure and flow resistance in this configuration of the stylet 226 and introducer 202.

The cannula 206 also includes a tapered distal tip 209, which functions to minimize possible damage to the distal end 208 of the cannula 206 during the tissue piercing and insertion procedure. The tapered distal tip 209 minimizes peel-back during the insertion procedure. Referring to FIG. 4a, in a particular embodiment, the taper angle 215 is between 20-25 degrees.

Referring to FIG. 4b, once the introducer 202 has been located at the target site, the stylet 226 is withdrawn from the introducer 202 and the RF probe assembly 106 is inserted into the introducer 202. In particular, the elongate shaft 184 is inserted into the cannula 206 until the active tip (including the energy delivery devices 192) at the distal end of the elongate shaft 184 extends beyond the distal end 228 of the cannula 206. The elongate shaft 184 has an outside diameter 236 such that a concentric clearance 238 between the elongate shaft 184 and the inside diameter of the cannula 206 is greater than 0.0010 inches. This concentric clearance 238 may be, for example, 0.0020+/−0.0010 inches, such as 0.0020 inches.

Referring to FIGS. 2 and 3, the introducer 202 may include a fluid introduction port 216 in the proximal hub 204 that is in fluid communication with the proximal end 210 of the cannula 206. This port 216 may be defined at a ninety-degree angle relative to a longitudinal axis of the introducer 202, as depicted particularly in FIG. 3. A flexible or rigid fluid delivery tube 218 can be connected to the port 216, and a fitting 220 may be connected to the opposite end of the tube 218, wherein fluids such as saline or a local anesthesia can be injected into the target tissue via the fitting 220 and port 216 while the RF probe assembly 106 remains inserted in the introducer 202. The tube 218 may be fixed to the port 216 and the fitting 220 with a suitable medical grade adhesive. The concentric clearance 238 between the elongate shaft 184 and the cannula 206 allows for flow of the fluid with minimal resistance. The fitting 220 may include a check valve that allows fluid to be injected into the fluid delivery port 216 through the fitting 220, for example with a syringe, while preventing backflow of fluid when the syringe is removed.

Referring to FIG. 5 in particular, a radiopaque element 222 may be fixed to the distal end 208 of the cannula 206 with an adhesive or other suitable fixation means. This radiopaque element 222 may have a tapered distal end and thus, defines the tapered distal tip 209 of the cannula 206. This element 222 may be co-extruded with the cannula 206 or coated onto the distal end 208. In addition a plurality of radiopaque bands or markers 224 may be applied over the polymer material at regular spaced intervals. The markers 224 and element 222 provide for visualization fluoroscopy in assessing the insertion depth of the introducer at the target site.

FIG. 5 also depicts a metal mesh or individual metal wires 225 around the outside of the polymer cannula 206 to add increased stiffness to the cannula 206. This metal material 225 may be embedded in the polymer material and extruded with the polymer tubing during manufacturing.

As discussed, the present invention encompasses an RF ablation probe system 200 (FIG. 1) for use in locating a radio frequency (RF) probe assembly 106 at a target location in tissue to treat or manage pain in a patient. The system 200 includes the RF probe assembly 106, the introducer 202 discussed above, as well as the stylet 226 that is insertable through the proximal hub 204 and into the cannula 206. The characteristics and features of the introducer 202 discussed above are applicable to the introducer 202 included with the RF ablation probe system 200.

The present invention also encompasses various method embodiments for use of the introducer and RF ablation probe system as described and enabled above.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A medical introducer for use in locating an energy delivery probe at a target location in tissue, comprising:
a proximal hub;
an elongate cannula having a distal end and a proximal end, the proximal end connected to the hub;
the cannula formed entirely of an electrically insulating polymer material; and
the cannula comprising a tapered distal tip.

2. The medical introducer of claim 1, wherein the hub is overmolded onto the proximal end of the cannula.

3. The medical introducer of claim 1, wherein the hub is bonded onto the proximal end of the cannula.

4. The medical introducer of claim 1, wherein the polymer material comprises a flexibility that allows the cannula to bend without kinking during insertion of the introducer into the tissue.

5. The medical introducer of claim 1, wherein the polymer material is one of nylon, fluorinated ethylene propylene (FEP), polytetrafluoroethylene (PTFE), high-density polyethylene (HDPE), polyamide, or polyether ether ketone (PEEK)

6. The medical introducer of claim 1, wherein the hub further comprises a fluid introduction port in fluid communication with the proximal end of the cannula.

7. The medical introducer of claim 1, further comprising a radiopaque element fixed to the distal end of the cannula, the radiopaque element defining the tapered distal tip of the cannula.

8. The medical introducer of claim 7, further comprising a plurality of radiopaque bands applied over the polymer material and spaced apart along the cannula.

9. An RF ablation probe system for use in locating a radio frequency (RF) probe at a target location in tissue, comprising:
an introducer, the introducer comprising
a proximal hub;
an elongate cannula having a distal end, a proximal end, and an inside diameter, the proximal end connected to the hub; and
the cannula formed entirely of an electrically insulating polymer material;
a stylet insertable through the hub and into the cannula, the stylet comprising a tissue-piercing distal end that extends from the distal end of the cannula when the stylet is inserted into the introducer, the stylet comprising an outside diameter such that a concentric clearance between the stylet and the inside diameter of the cannula is less than 0.0010 inches; and
an RF probe assembly comprising an elongate shaft insertable through the hub and into the cannula, the elongate shaft comprising an active distal tip that extends from the distal end of the cannula when the elongate shaft is inserted into the introducer, the elongate shaft comprising an outside diameter such that a clearance between the elongate shaft and the inside diameter of the cannula is greater than 0.0010 inches.

10. The RF ablation probe system of claim 9, wherein the cannula comprises a tapered distal tip;

11. The RF ablation probe system of claim 9, wherein the concentric clearance between the stylet and the inside diameter of the cannula is less than 0.0007+/−0.0002 inches.

12. The RF ablation probe system of claim 9, wherein the concentric clearance between the RF probe and the inside diameter of the cannula is 0.0020+/−0.0010 inches.

13. The RF ablation probe system of claim 9, wherein the hub is overmolded or bonded onto the proximal end of the cannula.

14. The RF ablation probe system of claim 9, wherein the polymer material comprises a flexibility that allows the cannula to bend without kinking during insertion of the introducer into the tissue.

15. The RF ablation probe system of claim 9, wherein the polymer material is one of nylon, fluorinated ethylene propylene (FEP), polytetrafluoroethylene (PTFE), high-density polyethylene (HDPE), polyamide, or polyether ether ketone (PEEK).

16. The RF ablation probe system of claim 9, wherein the hub further comprises a fluid introduction port in fluid communication with the proximal end of the cannula.

17. The RF ablation probe system of claim 9, further comprising a radiopaque element fixed to the distal end of the cannula, the radiopaque element defining the tapered distal tip.
